(19) United States
(12) Patent Application Publication
(10) Pub. No.: US 2014/0316297 A1
(43) Pub. Date: Oct. 23, 2014

(54) IMPEDANCE MONITORS, ELECTRODE ARRAYS AND METHODS OF USE

(71) Applicant: NONINVASIVE MEDICAL TECHNOLOGIES, INC., Las Vegas, NV (US)

(72) Inventors: Ann K. MCCAUGHAN, Las Vegas, NV (US); Marc O'GRIOFA, Las Vegas, NV (US); Philip HAMSKI, Las Vegas, NV (US)

(73) Assignee: NONINVASIVE MEDICAL TECHNOLOGIES, INC., Las Vegas, NV (US)

(21) Appl. No.: 14/186,757

(22) Filed: Feb. 21, 2014

Related U.S. Application Data
(60) Provisional application No. 61/768,011, filed on Feb. 22, 2013.

(51) Int. Cl. A61B 5/053

(52) U.S. Cl. CPC A61B 5/0537
USPC 600/547; 600/391

(57) ABSTRACT

A portable bioelectric impedance monitor for monitoring extracellular fluid levels includes a tetrapolar electrode array lead with four electrodes arranged sequentially and axially along the lead, and circuitry coupled with the at least four electrodes configured to measure bioelectric impedance extracellular fluid in a human subject at a frequency of less than 15 kHz. The electrodes are adhered to a human subject/patient on the patient's torso or one of the patient's limbs. One embodiment includes a Tetrapolar Analog Front End Patient Interface circuit configured to convert two electrode operation of a commercial Impedance Converter, Network Analyzer into a tetrapolar operation for excitation and impedance measurement of the human subject.

### CROSS REFERENCE TO RELATED APPLICATIONS

This application claims priority from U.S. Patent Application No. 61/768,011 filed Feb. 22, 2013, which, with U.S. Pat. No. 7,474,918, are incorporated by reference herein in their respective entireties

### BACKGROUND OF THE INVENTION

The present invention relates to the field of human bioelectric impedance measurement devices used to monitor a human patient or other human subject condition.

It is known in the art to measure human impedance to monitor levels of intrathoracic fluids, such as blood. In particular, it is known to use an impedance monitor to measure human thoracic impedance, along with electrocardiogram (EKG) signals, as indicative of blood flow and heart performance characteristics, as described in U.S. Pat. No. 5,443,073 (Wang et al.), the subject matter of which is incorporated by reference herein in its entirety. A portable device for non-invasive thoracic impedance measurement for the determination of Stroke Volume (SV) and Cardiac Output (CO) is described in U.S. Pat. No. 7,474,918. The relatively small and simple, portable, non-invasive device for bioelectric impedance measurement described in U.S. Pat. No. 7,474,918 was superior to numerous prior invasive and non-invasive thoracic impedance measurement devices and methods detailed in that patent.

It is further known that certain medical conditions, such as congestive heart failure (CHF) or renal disease, correlate qualitatively with the level and variation of the level of intrathoracic fluids.

It would further be useful to be able to monitor levels of tissue hydration in a human subject in real/near real time, in particular, extracellular fluid (ECF) levels, to gauge the subject's response to various interventions, for example, kidney dialysis.

### BRIEF SUMMARY OF THE INVENTION

According to one aspect of the invention, a device to monitor tissue hydration of a human subject comprises: at least four electrodes capable of being physically adhered and electrically coupled to the human subject; and circuitry coupled to four of the at least four electrodes to measure a bioelectric tissue impedance of the patient at a frequency of less than fifteen kilohertz. (<15 kHz).

In another aspect, the invention is a method of operating the aforesaid device to monitor extracellular fluid status of a human subject comprising the steps of: connecting four of the electrodes in a linear arrangement to the skin of the human subject; generating an oscillating voltage signal having a frequency of less than 15 kHz.; removing a dc bias from the oscillating voltage signal; converting the oscillating voltage signal into an oscillating current having a frequency of less than 15 kHz; passing the oscillating current through the human subject between a first pair of the electrodes; sampling voltages from the human subject through a second pair of electrodes positioned between the first pair of electrodes on the human subject; generating a differential voltage signal from sampled voltages; converting the differential voltage signal into an alternating current; adding to the alternating current a constant bias equivalent to the dc bias removed from the oscillating voltage signal to provide a current output; and determining from the current output one or more biometric impedance values for the human subject.

In yet another aspect, the invention is a method of monitoring extracellular fluid status of a human subject, the method comprising: adhering to skin of the human subject four spaced apart electrodes in a linear array; passing an oscillating current having a frequency of less that fifteen kilohertz through the patient between an outermost pair of the four electrodes; sensing voltage levels from the human subject through an innermost pair of the four electrodes; calculating a bioelectric impedance value for the human subject from the sensed voltage levels; and outputting the calculated biometric impedance value to a human interface device.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 depicts a prior art impedance monitor of U.S. Pat. No. 7,474,918 connected to a patient/user for thoracic impedance measurement;

FIG. 2 is a front perspective view of the front face of a base unit of the monitor of FIG. 1,

FIG. 3A is a perspective view of an electrode array assembly of the impedance monitor of FIG. 1, in accordance with a first preferred embodiment of the present invention;

FIG. 3B is a plan view of a first side of an electrode pad assembly of the electrode array assembly of FIG. 3A;

FIG. 3C is a plan view of a second side of an electrode pad assembly of the electrode array assembly of FIG. 3A;

FIG. 3D is a plan view showing the electrode pad assembly of FIG. 3B separated from first and second electrodes of the electrode array assembly of FIG. 3A;

FIG. 3E is a perspective view of components of an electrode array assembly in accordance with a second preferred embodiment of the present invention;

FIG. 4 is a block diagram of the major circuit components of the prior art thoracic impedance monitor base unit;

FIG. 5 is a diagram of steps of a method of monitoring thoracic fluid level of a person in accordance with the present invention;

FIGS. 6A-6D are flow diagrams describing in detail operation of the prior art base unit;

FIG. 7 is a functional block diagram of the circuit components of the base unit modified for Extracellular Fluid (ECF) patient monitoring;

FIG. 8 is a functional block diagram of the circuit components of the base unit further modified from FIG. 7 to selectively provide for Extracellular Fluid (ECF) or conventional thoracic impedance patient monitoring, as desired;

FIG. 9 is a functional block diagram of the circuit components of the base unit further modified from FIG. 8 to incorporate an Impedance Converter and Network circuit operating through a Tetrapolar Front End Patient Interface;

FIG. 10 is a functional block diagram of the components of the Impedance Converter and Network circuit used with Tetrapolar Front End Patient Interface of FIG. 9;

FIG. 11 is a functional block diagram of the components of the Tetrapolar Front End Patient Interface of FIG. 9 used with the Impedance Converter and Network circuit of FIGS. 9 and 10;

FIG. 12 illustrates one possible use of any of the subject systems to measure Extracellular Fluid in a human subject; and

FIGS. 13-16 are detailed circuit diagrams for an embodiment of the Tetrapolar Analog Front End Patient Interface of FIG. 11.

### DETAILED DESCRIPTION OF THE INVENTION

In accordance with the present invention, a process for monitoring human subjects such as medical patients comprises the steps of applying electrodes to points on the body of the subject/patient, passing an alternating current of very low amperage between a first pair of the electrodes, measuring voltages (V) of the body through a second pair of the electrodes located on the subject/patient between the first pair, calculating an average impedance value Zo based on the applied current (I) and measured voltages (V) and displaying the average impedance value for comparison with baseline values previously established preferably when the subject/patient was in a known, stable condition, to determine if differences are within established tolerances.

The process is preferably carried out with a battery powered, portable base unit which performs all the necessary functions. A description of the prior art U.S. Pat. No. 7,474,018 thoracic impedance monitor is first given as the present invention uses or incorporates many of the same features and operations. The invention of the prior art U.S. Pat. No. 7,474,018 resided in an “early warning” monitoring system or “monitor” and a method for determining changes in the status of patients with chronic congestive heart failure (CHF) by measuring intrathoracic fluid, with the goal of intervening before the onset of acute congestive heart failure.

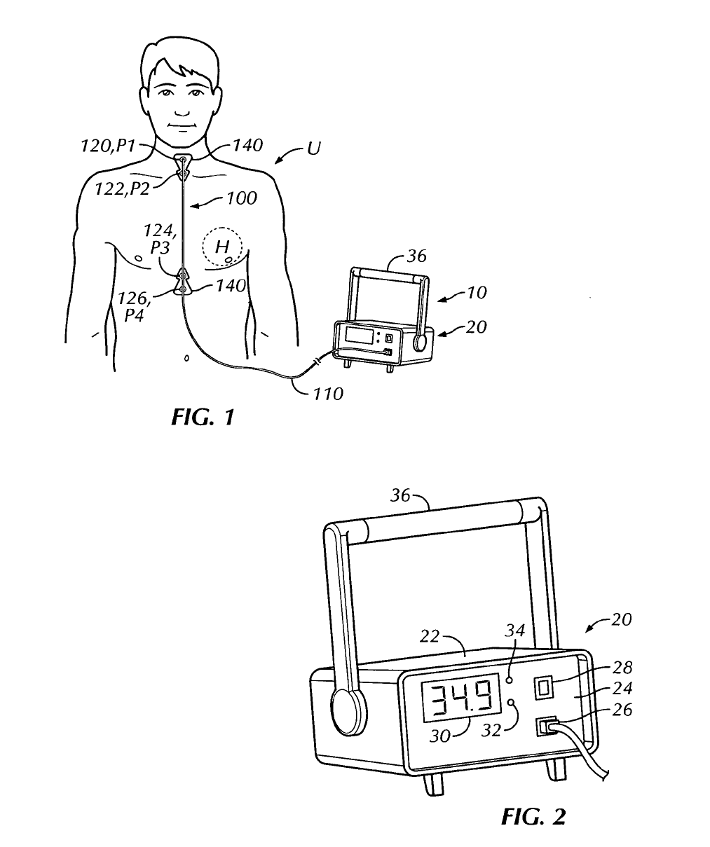

FIG. 1 depicts the prior art, non-invasive, bioelectric impedance monitoring system or “monitor” of U.S. Pat. No. 7,474,018, which is indicated generally at 10. The system is shown installed on a patient or other user U to measure thoracic impedance using a patient interface that includes a four electrode array assembly 100 which is coupled to a small, hand transportable base unit 20.

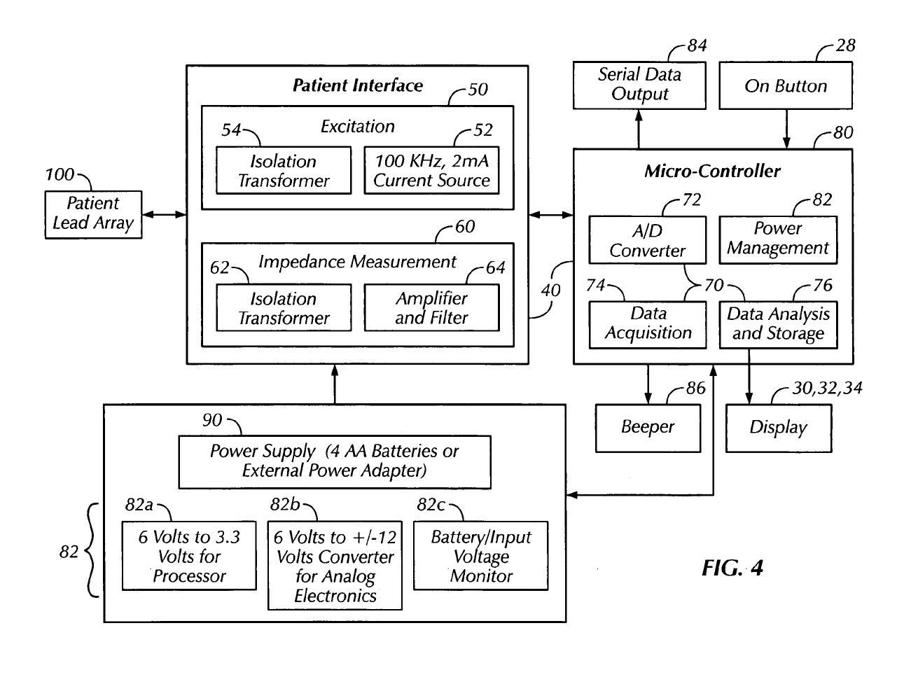

FIG. 2 depicts a front panel 24 of the base unit 20. The base unit 20 is relatively lightweight (about one pound or one-half kilo) and is contained in a relatively small (i.e. hand transportable) housing 22. Preferably, the base unit 20 is provided with a handle 36 to facilitate transport. The base unit 20 contains several user interfaces in addition to a connector port 26 for receiving a connection end of the electrode array assembly 100. These interfaces were a three digit display 30 (e.g. formed by three, seven segment LED's) which preferably digitally display impedance as xx.x ohms, a start switch 28 to start the system 10, a low battery alert light 32, and a cable disconnect alert light 34. Preferably the base unit 20 also contained a beeper 86 (see FIG. 4) or other sound generator for signaling purposes. Alternatively, additional alert lights (not illustrated) could be substituted for the beeper 86.

The base unit 20 was configured to perform all necessary steps to measure, determine and display the patient's base thoracic impedance after the start switch 28 is actuated. However, the system 10 did not provide any patient diagnostic parameters. That is, it provided only a measurement of impedance over a predetermined fixed length of the patient's body. This value can be compared with other impedance values for the patient or against limit values. The information provided by system 10 would be evaluated along with various other parameters by health care or other professional to identify the use of the information for their specific purpose.

The base unit 20 provided the following outputs. The three digit LED display 30 preferably displayed impedance value as xx.x. During measurement, a rotating/flickering pattern was displayed to indicate the measurement is in progress. To ensure that the user U records ONLY the impedance values, the system software preferably did not display any numerical values other than impedance value. This means that there were no countdown timers and no error or diagnostic codes expressed as numerical values. The base unit 20 would also indicate an error condition (by the beeper or flashing lights) in the event it detects that it could not perform a valid impedance measurement or that the impedance value was outside of a predetermined measurement range (suggestedly 5 to 55 ohms). If the electrode array assembly was disconnected from the system cable disconnect alert light 34 would illuminate.

The base unit 20 would activate the low battery indication light 32 in the event it detected that the battery voltage is below a level that will allow for reliable impedance measurement. In the event of a low battery voltage condition, the base unit 20 might blink this LED 32, for example at a rate of once every 10 (+/−0.5) seconds for a period of 30 (+/−2) sec. If the battery voltage dropped below 5.25 volts, but remains above 4.75 volts, the impedance results would still be displayed along with blinking battery condition LED 32 to indicate that the battery power was getting low but still acceptable. If the battery voltage dropped below 4.75 volts, both LEDs 32, 34 would be made to blink to indicate that the battery voltage was low and accurate results could not be displayed. Preferably a micro-controller 80 in the base unit 20 would continue to operate below 4.75 volts, even though an accurate measurement could not be made, to warn the user of the condition of the unit.

The base unit 20 could be configured to provide various beeper alerts to the user. Preferably the base unit 20 beeped to indicate that the measurement is completed and the displayed value should be recorded. The beeper 86 could further be activated to indicate other, different conditions or steps, for example, when the base unit 20 was initially activated, while the unit was initializing, while the power supply was stabilizing, while measurements were being taken and/or before the unit shut itself off. The beeper 86 could also be activated in the event a successful measurement was not accomplished or an error condition was detected. It was suggested that different beep patterns could be used for different conditions including different states of the base unit 20.

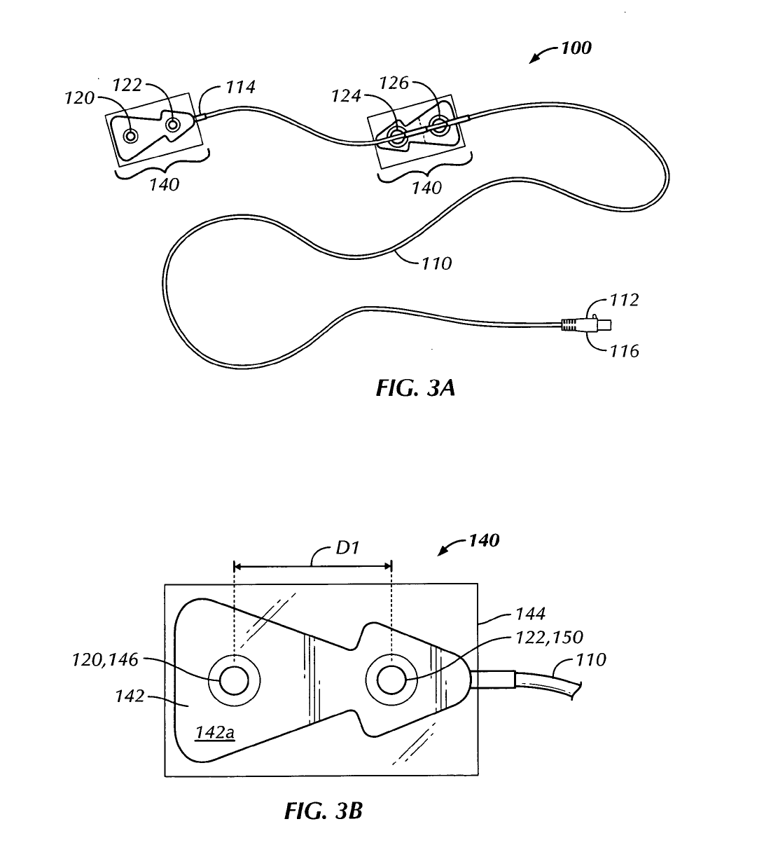

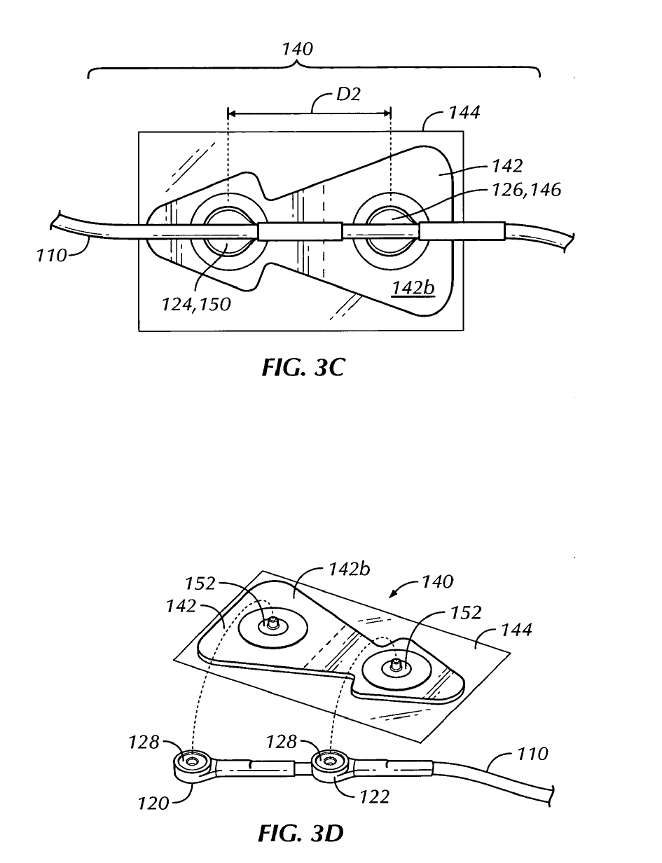

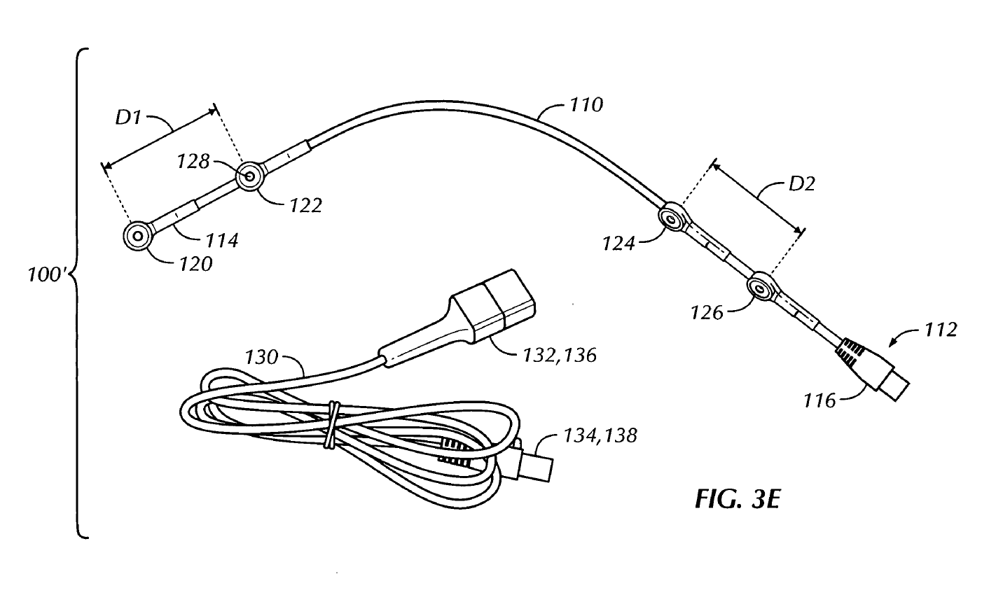

Referring to FIGS. 3A-3D, a first preferred embodiment of the electrode array assembly 100 included a single, linear electrode array lead 110 having a first end 112 and a second end 114. An electrical connector 116 is provided at the first end 112. Electrical connector 116 operatively connects to connector port 26. First through fourth electrodes 120, 122, 124, and 126 are arranged axially and spaced along the length of the lead 110. As discussed further below, preferably first and fourth electrodes 120, 126 are current sources, while preferably second and third electrodes 122, 124 measure electrical potential. Because the electrodes 120-126 are fixed along the lead 110, their spacing relative to one another is also fixed and predetermined, with the first and second electrodes 120, 122 being spaced a first pre-determined distance D1, and the third and fourth electrodes 124, 126 being spaced an equal pre-determined distance D2. The pre-determined distances D1, D2 were preferably about five centimeters or about two inches.

Preferably, identical first and second electrode pad assemblies 140 were releasably connected to the electrodes 120-126. The preferred electrode pad assemblies included an overlapped arrow-shaped body member 142 into which were mounted a first electrode pad 146 and a second electrode pad 150. The body member 142 had a first side 142a, and the electrode pads 146, 150 were exposed on this first side 142a. On a second side 142b of the body member, male snap elements 152, rigidly connected to the electrode pads 146, 150, are exposed. The male snap elements 152 were adapted to releasably connect with complementary female snap elements 128 provided in the electrodes 120-126 on the lead 110. Any other conventional structure used for coupling electrode pads to such cardio leads could also be used.

Preferably, the body member 142 was pre-coated during manufacture with a contact adhesive on the first side 142a. A removable, adhesive protective film 144 was preferably provided. Preferably, the electrode pads 146, 150 were coated with an electrically conductive hydrogel which acted along with the contact adhesive and allowed the electrode pads 146, 150 to releasably adhere to the user's skin. The electrodes 120-126 and electrode pads 146, 150 incorporated into the electrode array assembly 110 were off-the-shelf commercially available components.

Referring to FIG. 3E, a second embodiment electrode array assembly 100′ was generally similar to the first embodiment electrode array assembly 100, with the exception that a second embodiment electrode array lead 110′ was substantially shorter, and a connection cord 130 was provided to connect the electrode array lead 110′ to the base unit 20. The connection cord 130 had a first end 132, a second end 134, a first connector 136 at the first end 132 configured to mate with array lead connector 116, and a second connector 138 at the second end 134 configured to mate with base unit connector port 26. Note that electrode pad assemblies 140 are omitted from the illustration of FIG. 3E, but conventional conductive pads were used as part of the second embodiment electrode array assembly 100′.

Each of the array leads 110, 110′ was flexible along its length. While the spacing between the first and second electrodes 120, 122 and between the third and fourth electrodes 124, 126 with the electrodes 120-126 operatively connected to a user was preferably the same for all users, given the flexibility of the array lead 110, 110′, the spacing between the second electrode 122 and the third electrode 124 could be adjusted to accommodate users of various sizes. That is, for a user having a long sternum, with the electrodes 120-126 placed as indicated above, the electrode array lead 110, 110′ will be more fully extended between the second and third electrodes 122, 124 than would be the case for a user having a shorter sternum and also having the electrodes 120-126 placed as indicated above.

With reference now to FIG. 4, a block diagram of the circuitry 40 of the prior art thoracic impedance base unit 20 of U.S. Pat. No. 7,474,918 included signal generating circuitry 50, voltage detection circuitry 60 and impedance calculation circuitry 70. The impedance calculation circuitry 70 included an analog/digital converter 72, data acquisition circuitry 74, and data analysis and storage circuitry 76. Along with power management circuitry 82, the impedance calculation circuitry 70 was provided by a micro-controller 80.

The signal generating circuitry 50 generated the stable excitation current (I). A current source subcircuit 52 included a constant current source (not depicted) and clock oscillator (not depicted) to supply a current of about 2 mA or less, preferably a 1.98±0.01 mA, at a 100±10 kHz (i.e., about 100 kHz) frequency preferably to the first and fourth electrodes 120, 126 through an isolation transformer 54, the connection cord 130 and electrode array lead 110. The current source subcircuit 52 is configured to output a current of less than 4 mA under all conditions including equipment component failure. The wave form of the current was suggestedly sinusoidal with less than ten percent total harmonic distortion. Voltage values across the second and third electrodes 122, 124, were passed through isolation transformer 62 to an amplifier and low pass filter subcircuit 64. The low pass filter subcircuit 64 functioned to remove extraneous electrical interference from ambient sources, for example, home appliances operating on standard residential 60 Hz current. A preferred cut-off frequency of the low pass filter subcircuit 64 was about 50 Hz. The base unit 20 measured voltage developed across detection electrodes 122, 124 when the excitation current source was energized. The voltage level would be between about 18 millivolts and 104 millivolts (to provide an anticipated range of impedance measurement of about 10 ohms to 50 ohms, at the 2 mA current).

Micro-controller 80 controlled generation of the excitation current and received the filtered voltage analog signal from the amplifier and low pass filter 64 at the input of the analog to digital converter 72. Suggestedly the injected current was not generated for a short period of time (e.g. fifteen to thirty seconds) after the start switch 28 was actuated to allow the user to settle into a quiescent state. The current was then injected for a predetermined period, e.g. thirty seconds, to perform the measurement. Voltage values sampled from the A/D converter 72 were received by the data acquisition circuitry 74 of the micro-controller 80 suggestedly at a rate of about five samples per second for all or most of the thirty second period. Data analysis and storage circuitry 76 of micro-controller 80 summed the counts generated by the A/D converter 72, divided the sum by the total number of samples taken to provide an average voltage value which was converted into an impedance value. The algorithm used for generating impedance in tenths of ohms was: averaged A/D counts*Gain+Offset, where in the preferred circuit the Gain was 0.6112 and the Offset was 1.1074. Gain and Offset were based on the electronics design and operating range and were used for all base units 20. Each system 10 was calibrated to match the use of these numbers. The data analysis circuitry 76 also controlled the various displays 30, 32, and 34. The power management circuitry 82 controlled the generation and distribution of power in the base unit circuitry 40 to control operation of the system 10. Specific functions of the power management circuitry 82 included a first function 82a of providing power to the processor; a second function 82b of providing power to the A/D converter, and a third function 82c of monitoring the input voltage.

As indicated, a power supply 90 could be provided by conventional dry-cell batteries or by an external power adapter connected to a conventional 120 V outlet.

The base unit 20 could be provided with a serial port 84 to work with logic level signals. The timing for the serial data can be similar to RS232 signal or other conventional data transfer format. The base unit 20 would preferably be provided with a serial port, for example one configured to operate at 9600 baud, with 8 bit data, 1 Start bit, 1 Stop bit and no parity bit format. An external level translator could be necessary to interface the base unit to a PC or a PALM device. Upon receipt of a specific command, the base 20 unit would be configured to transmit the information related to all or a subset (e.g. the last ten) of the readings of the impedance measurement. This information may have also included the date and time of measurement, impedance value, and/or the serial number of the unit.

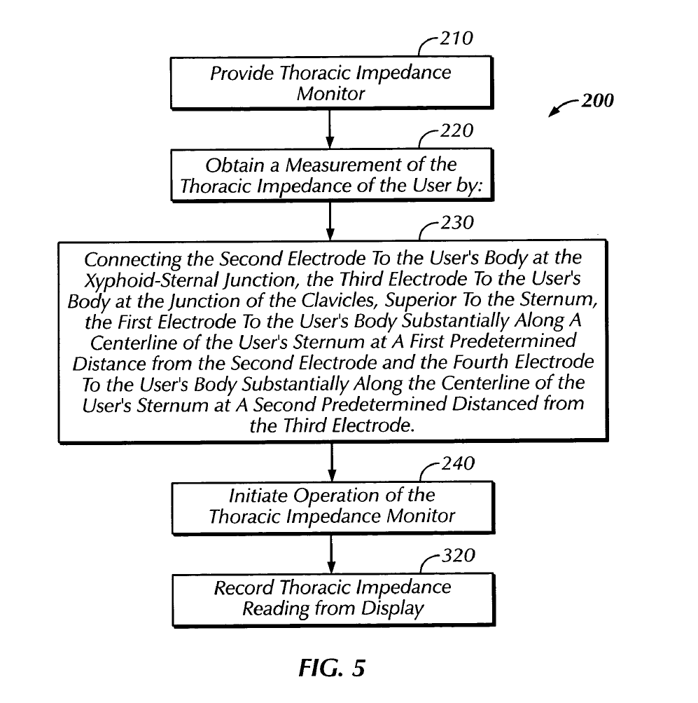

With reference to FIG. 5, a method of monitoring thoracic fluid level of a person included a first step 210 of providing the thoracic impedance monitor 10, as described herein. In a second step 220, the user obtained a measurement of their thoracic impedance. To accomplish this second step 220, in a third step 230, the user connected the first through fourth electrodes 120-126, via electrode pads 146, 150, to the users' body, as described above.

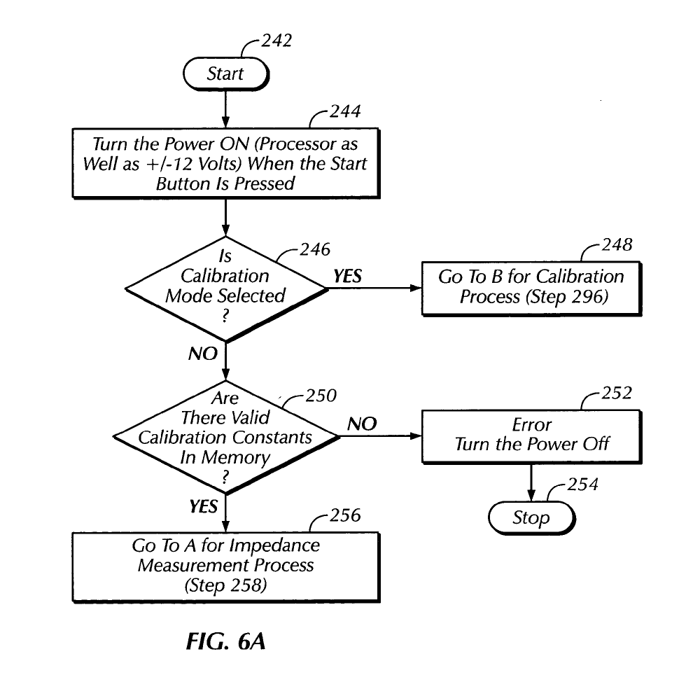

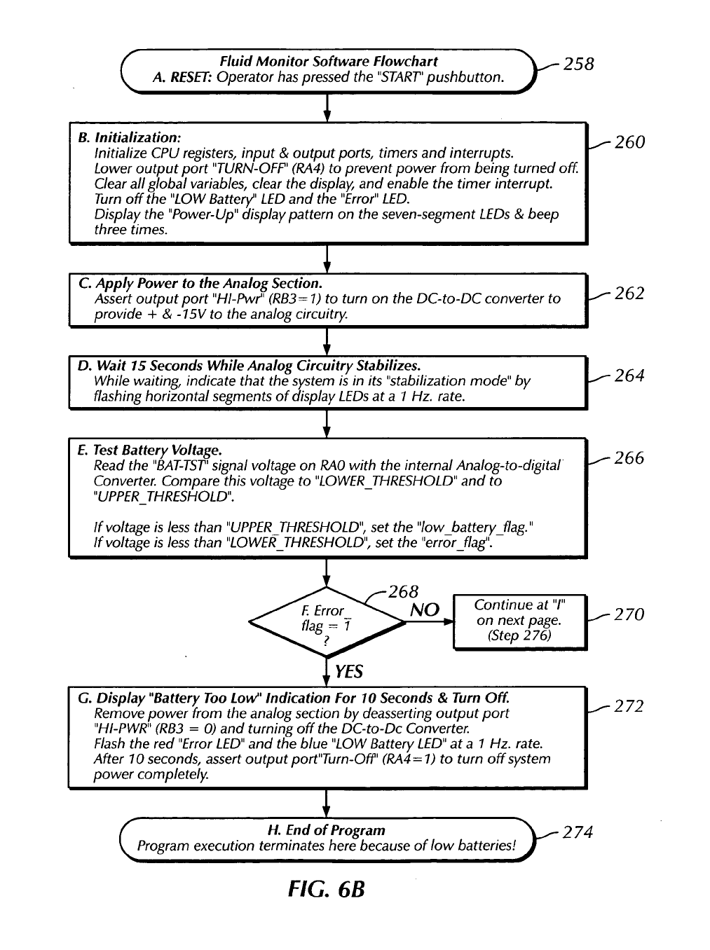

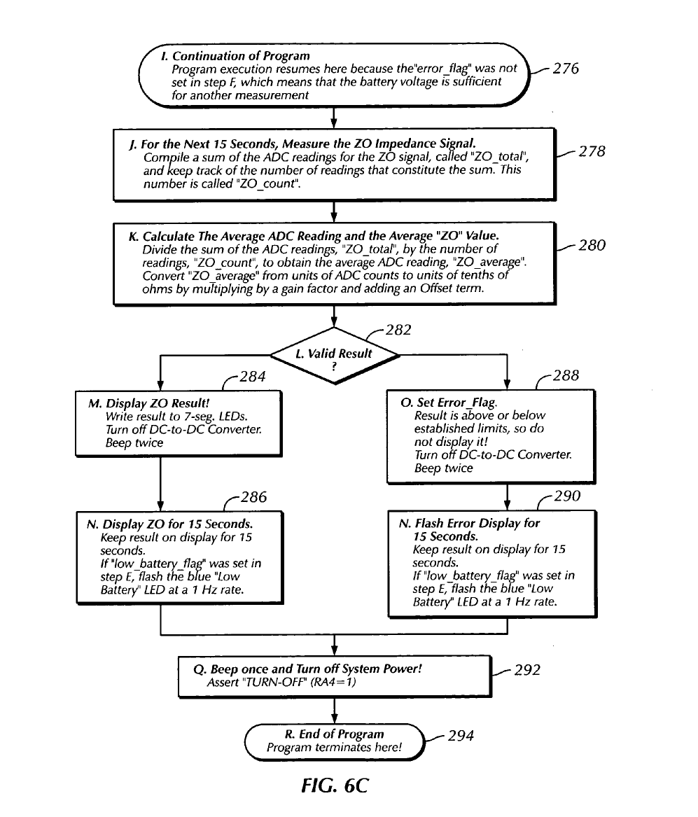

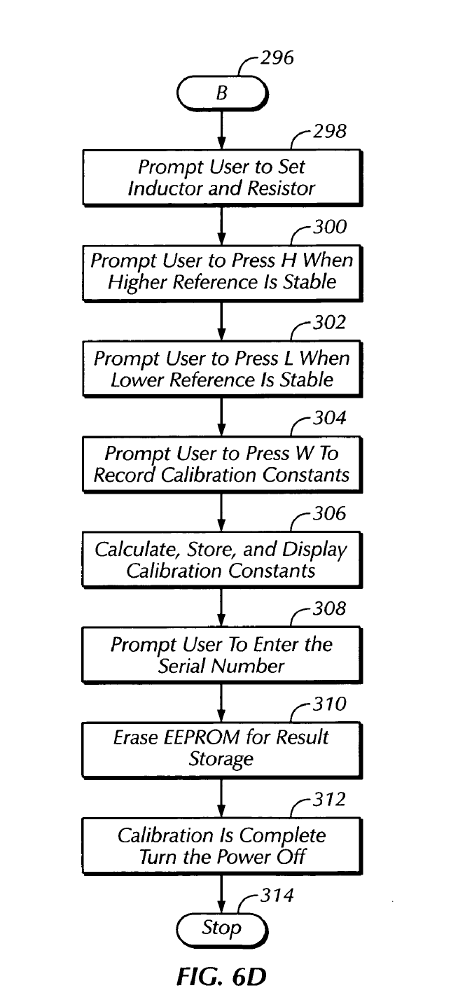

With the electrodes 120-126 in place, in a fourth step 240, the user initiated operation of the impedance monitor 10 by actuating the start switch 28. The user was to remain “relatively” still for the length of the measurement period. The system 10 injected the relatively high frequency (e.g. about 100 KHz) very low amperage (about 2 or less mA) current into the user and took voltage readings from the second and third electrodes 122, 124 for a period of time (e.g. about thirty seconds), calculated the average thoracic (base) impedance and then displayed the average value, preferably for a predetermined period (e.g. fifteen seconds to two minutes). In particular, activation of the start switch 28 initiated a series of steps 242-314. For brevity, the reader is referred to FIGS. 6A-6D, which describe in detail the series of steps 242-314. In short, assuming proper functioning of the impedance monitor 10, activation of the start switch 28 culminated in display of the user's thoracic impedance (measured in ohms) on the base unit display 30. Once the reading was obtained, in a fifth step 320, it was desirable that the user log the reading into a record of impedance measurements taken over time.

Preferably, the user need use the system 10 only once a day for thoracic impedance but might take it more than once a day if needed or desired. The total time required for a test was brief, approximately five minutes. Preferably, to improve the ability to compare measurements, the measurements were to be taken at the same time of day (thoracic impedance measurements typically vary over the course of a day, as eating, drinking, and other activities affect thoracic fluid levels). More preferably, the test was performed daily before the user ate his or her first meal of the day. The test might be taken more often, for example, to monitor the effects of medication (e.g. diuretics) or exercise.

It has been found that the basic thoracic impedance monitoring device described above could be modified and used in different ways to better monitor relative fluid levels in human patient tissues. More particularly, it has been found that a relative hydration status of a human subject such as a patient can be based on the impedance values (Z) reported in ohms over different ranges of frequency measurements. Extracellular Fluid (“ECF”), sometimes referred to as Extracellular Water (“ECW”), is the fluid which surrounds cellular membranes in human tissue. Intracellular Fluid (“ICF”), sometimes referred to as Intracellular Water (“ICW”), is the fluid trapped in the cellular membranes forming human tissue. The ECF/ECW and ICF/ICW are predominately electrical resistive entities, whereas the cellular membrane, due to its lipid layer, has an isolating (capacitive) behavior. It has been found that the behavior of an injected current will be different for “low” and “high” frequencies. Low frequency currents only flow around the cells through the ECF/ECW, whereas high frequency currents will also pass through the cell membrane and the ICF/ICW. Thoracic impedance measurement is therefore a measure of the two. “Low Frequency” is hereinafter used to refer to a bioelectric impedance measuring current of a sufficiently low frequency magnitude as to flow only or essentially only through Extracellular Fluid component in the tissue of a human subject. A “Low Frequency” impedance measuring current is suggestedly less than 15 kHz (<15 kHz), preferably less than 10 kHz and, more preferably, only about 5 kHz. “High frequency” is hereinafter used to refer to an impedance measuring current of a sufficiently high frequency magnitude as to flow through or essentially through both the Intracellular (ICF) and Extracellular (ECF) fluid components in the tissue of a human subject. A “High Frequency” impedance measuring current therefore above 15 kHz (>15 kHz) and even above 50 kHz (>50 kHz) and more typically about 100 kHz like that of the described U.S. Pat. No. 7,474,918 device. The clinical benefit resides in the serial determination of these ECF/ECW impedance values as the patient undergoes therapeutic interventions as a gauge of the relative changes in the EC fluid volumes, for example, during dialysis treatment.

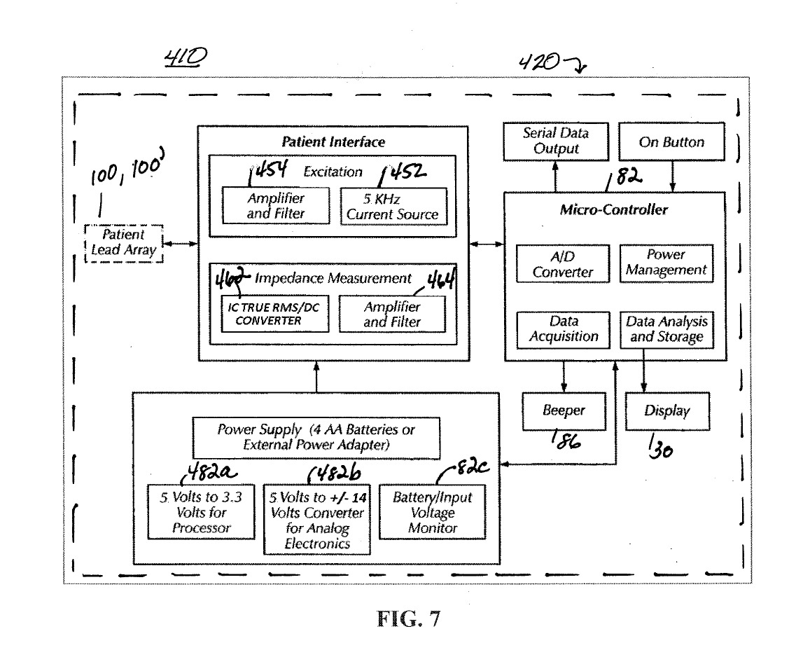

Referring to FIG. 7, a modified system 410 with modified Patient Interface has been substituted for that of FIG. 4 to simplify the design and to enable measurement of ECF/ECW generated impedance values. Apart from the Patient Lead Array, which remains the same, the other components of the system would again be housed in a base unit 420. In particular, a 5 kHz signal source 452 has been substituted for the original 100 kHz signal source 52 and appropriate operational amplifiers with appropriate filter(s) 454, 464 have been substituted for the original isolation transformers 54, 62 and amplifier/filter 64. In addition, an RMS to DC converter IC chip 462 has been provided as a detector for front end impedance measurement to reduce the computational load on the main micro-controller 80. There has been slight revisions to characteristics of the power supplies 482a, 482b reflective of the changes to the patient interface circuitry. No changes to the software of microcontroller 80 were need to continue to drive the display 30 to output impedance values in decimal form or to operate the sound output/beeper 86.

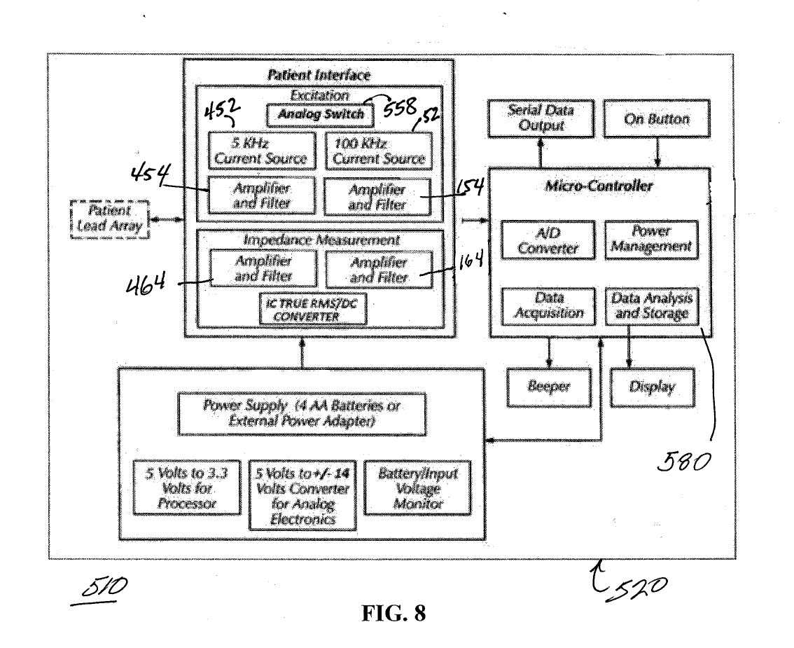

FIG. 8 represents a further modification of the FIG. 7 system (or alternate revision of the original FIG. 4 device). The FIG. 8 system 510 permits either conventional thoracic impedance measurements with a 100 kHz signal (current) source 52 and circuitry or ECF/W impedance measurements with a low frequency (5 kHz) signal (current) source 452 and related excitation and measurement circuitry. Again, apart from the Patient Lead Array 100, the other components would be housed in a base unit 520. CMOS analog switch circuitry 558 was provided to control a DPDT switch via the micro-controller 580 for user selection of the current source. Existing coding of the micro-controller was modified to permit storage and use of two sets of calibration gain and offset figures, with the appropriate figures being used automatically based upon the current source selected by the user.

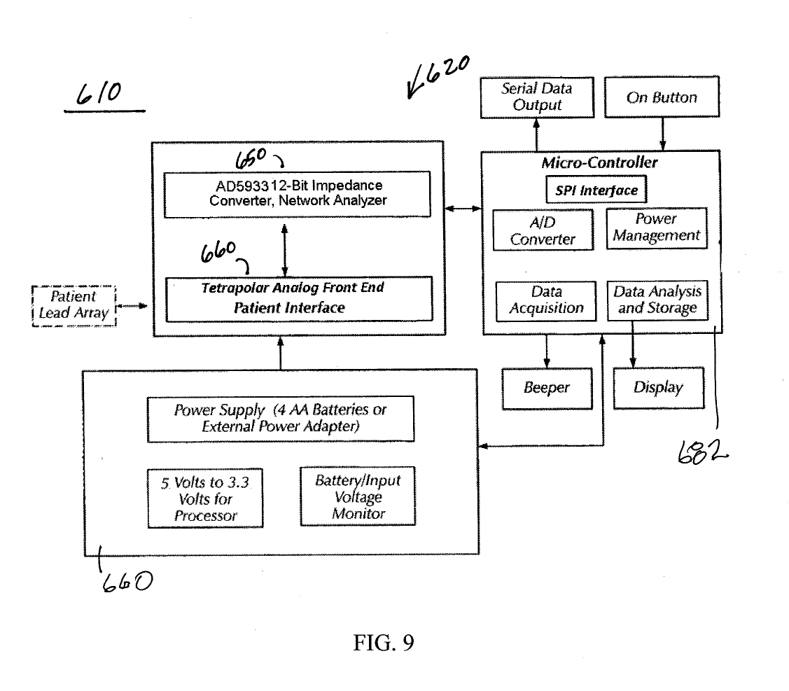

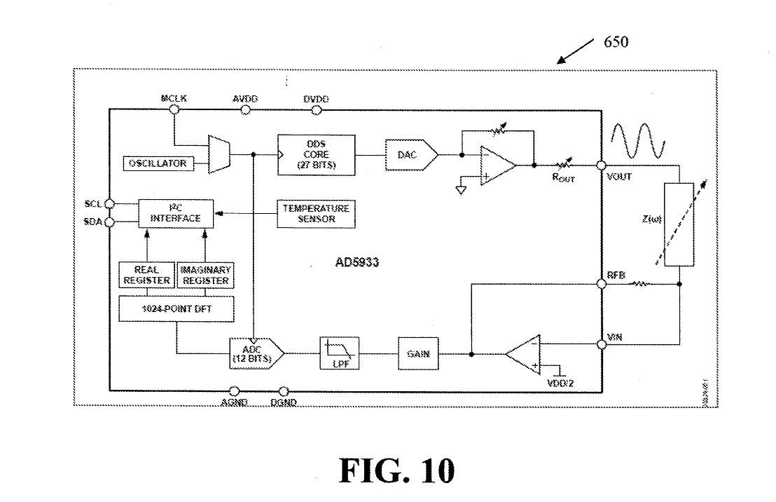

FIG. 9 represents a further improvement to the Low Frequency system of FIG. 7. Again, apart from the Patient Lead Array, the components of this system 610 are housed in a base unit 620. Here, a commercial circuit, an Analog Devices AD5933 1 MPSP, 12 Bit Impedance Converter, Network Analyzer 650 is provided as a current generator, voltage receiver and impedance data processor. A functional block diagram of AD5933 Impedance Converter, Network Analyzer 650 is set forth in FIG. 10. The AD5933 circuit 650 has an output or transmit stage generating and providing at VOUT, an excitation signal at a particular frequency to an external impedance (i.e. the patient/subject) to be measured. It has an input or receive stage that samples at VIN, the excitation signal after it has been passed through the external impedance. The input stage comprises a current-to-voltage amplifier, followed by a programmable gain amplifier, anti-aliasing (low pass) filter, and an analog to digital converter (ADC). Output of the ADC is passed to an on-board DSP engine with discrete Fourier transform algorithm which outputs calculated real (E) and imaginary (I) data-words. These words are passed from the AD5933 circuit to the microprocessor controller 680 for calculation of impedance (Z) values.

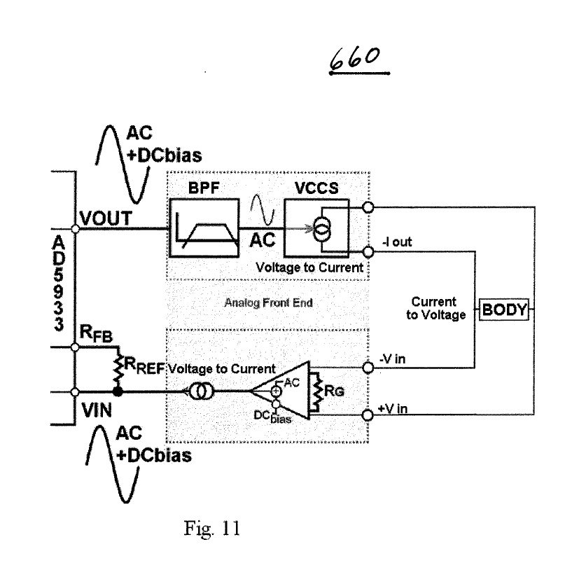

The AD5933 circuit 650 is used in combination with a Tetrapolar Analog Front End Patient Interface 660, a functional block diagram of which is presented in FIG. 11. As can be seen, the Tetrapolar Analog Front End Patient Interface 660 is connected across the VIN, VOUT, RFB connection points of the AD5933 circuit 650. Tetrapolar Analog Front End Patient Interface 660 converts the bipolar impedance operation of the AD5933 circuit 650 circuit into tetrapolar operation. The AD5933 circuit 650 is configured to operate as a two-electrode impedance measurement device and this fact limits severely the range of application of usage, e.g. applications of spectral characterization are basically discarded since the impedance measurement obtained will also contain the electrode polarization impedance as well as the electrode-skin impedance. Another important limitation of AD5933 circuit 650 is a safety issue. The voltage output VOUT contains a dc level component, a dc bias. This imbalance produces a dc voltage across the electrodes and the body, introducing dc current into the body of any human subject on which it might be used, which can be a health hazard for the subject. The AD5933 circuit 650 is a voltage-driven measurement system that does not itself provide any control over the injected current. This is a separate safety hazard issue since the injected current can be larger than recognized limits, for example, the limits set by the International Electrotechnical Commission standard IEC-60601 for electrical medical equipment. For these many reasons, the AD5933 circuit 650 is itself unsuitable for human bioelectric impedance or tetrapolar impedance determination.

The four terminal, Tetrapolar Analog Front End Patient Interface 660 provides an interface between the AD5933 circuit 650, and the human subject. As such, it must have the proper input and output stages to interconnect to each of them.

The four terminal, Tetrapolar Analog Front End Patient Interface 660 may be considered as a combination of two voltage-to-current converters, one in the direction from AD5933 circuit 650 to the human subject's body and another from the human subject's body to AD5933 circuit 650. Since AD5933 circuit 650 applies voltage at its VOUT output and expects a current flowing into its VIN input, the four terminal Tetrapolar Analog Front End Patient Interface 660 interfaces with AD5933 circuit 650 has a voltage input and a current output. The current source output generates the current resulting from the ratio of VOUT and the impedance of the body, which is the current expected by AD5933 circuit 650 at the VIN input. At the body side, the four terminal Tetrapolar Analog Front End 660 provides a current source as output while the input is a differential voltage measurement channel. The current source excites the human subject with an alternating current. In this case, an output current of 900 μA rms has been selected to fully comply with IEC-60601 for electrical safety, but that level is only currently preferred and is neither fixed nor required for measurement purposes.

The operation of the four terminal Tetrapolar Analog Front End Patient Interface 660 can be described as follows. The AC voltage output (VOUT) of the AD5933 circuit 650 is passed to the input of the first voltage to current converter, which includes at least a High-pass filter (HPF) providing first order filtering at 500 Hz for bias removal and 60 Hz suppression. It may also be passed through a Low-pass filter (LPF) for second order filtering at 1000 Hz. The HPF or the combined HPF/LPF may be replaced by other types or notch or Band-pass filter (BPF). The filtered AC voltage (Vac) drives a voltage-controlled current source (VCCS) of the first voltage to current converter, which injects an AC current (+I or Iout) into the body of the human subject. I+/Iout is directly proportional to the Vac, the filtered VOUT. The AC current I+/Iout causes a voltage drop across the body of the human subject, which is sensed by the second voltage to current converter. Since the voltage drop at the body of the human subject drives the second voltage to current converter, it generates an AC current proportional to the voltage drop in the body of the human subject. Finally, a DC component is added to the generated AC current. This added DC component is equivalent to the DC bias originally removed from VOUT. The resulting alternating current is fed to the VIN and RFB connections of the AD5933 circuit 650.

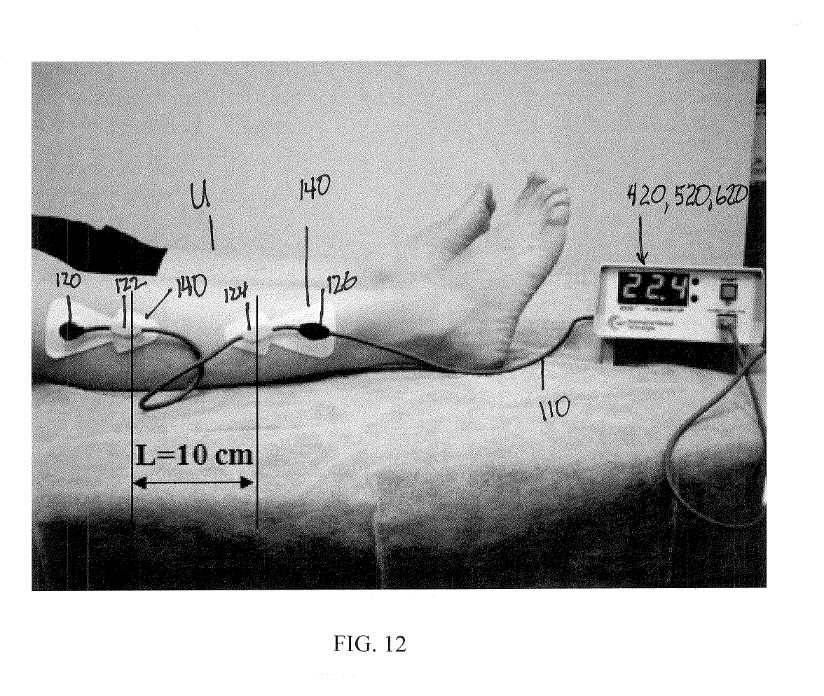

FIG. 12 illustrates the use of any of the systems 410, 510, 610 with a human subject. The electrode pad assemblies 140 of the single, linear electrode array lead 110 are adhered to the patient U. A suggested spacing between the inner (voltage) electrodes is about ten centimeters, but different lead arrangements and different electrode spacings might be used. The electrodes are applied linearly to the patient U but may be applied to either the torso of the patient as in FIG. 1 or to a limb. In FIG. 12, the electrodes are applied to a human subject's leg, where they might be used to monitor ECF fluid level changes in a patient undergoing dialysis.

Thereafter, the unit 410, 510, 620 generates and feeds a Low Frequency, low amperage current between the outer two electrodes 120, 126 and takes voltage measurements across the inner pair of electrodes 122, 124. An impedance value is calculated by the unit 410, 510, 610 and uploaded to the display 30. Individual measurements may be taken at spaced time intervals and displayed or series of measurements may be made and combined in various ways, for example, averaged non-overlapping or overlapping serial blocks of measurements. The real time/near real time reaction of the patient/subject to a procedure such as dialysis can be monitored by observing the changes in measured impedance values on the display.

It will be appreciated that measurement of ECF/ECW differs from thoracic impedance measurement for cardiopulmonary purposes by (1) the use of a Low Frequency signal and (2) the ability to locate the electrodes anywhere on the torso or any of the limbs of the human subject. Limb location is actually preferred for certain applications such as ECF monitoring of dialysis patients as illustrated by FIG. 12.

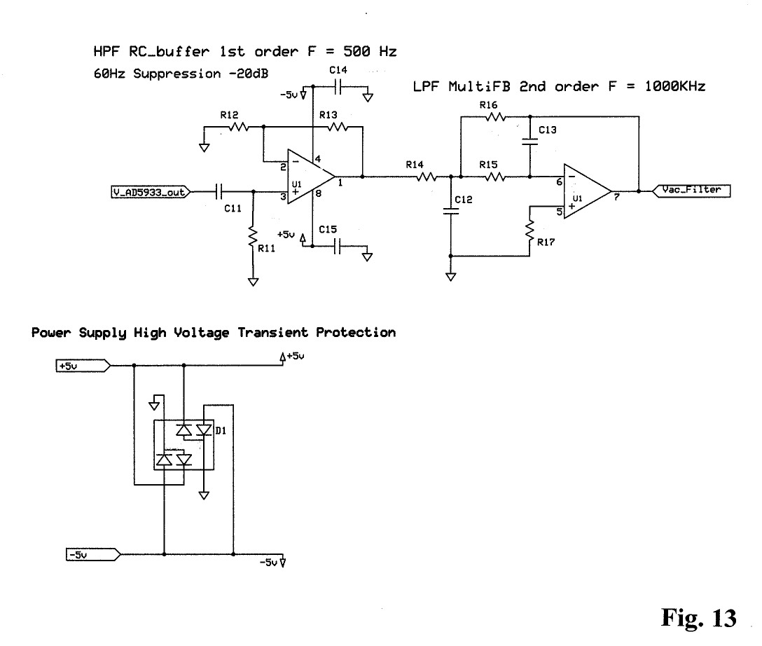

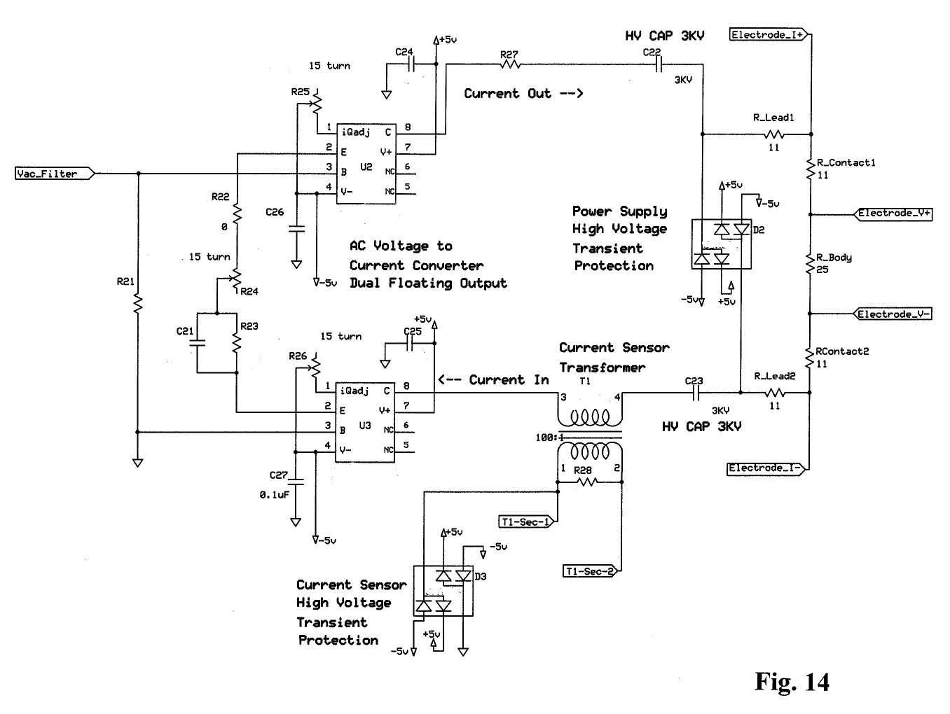

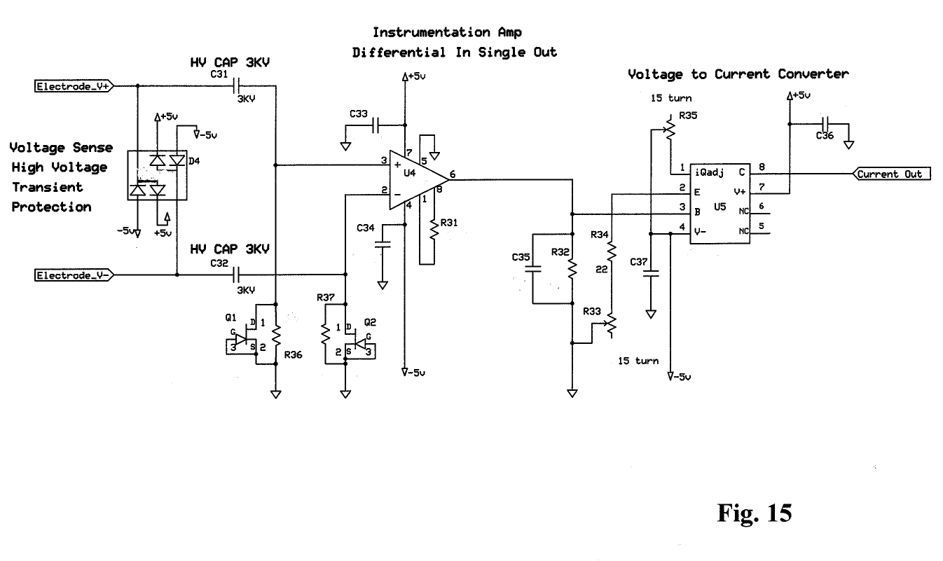

FIGS. 13-16 are detailed diagrams of components for a suggested Tetrapolar Analog Front End Patient Interface 660. FIG. 13 provides details of the filtering subcircuit associated with the first voltage to current converter. The signal from the VOUT terminal of the AD5933 circuit 650 is passed through a High Pass Filter and then a Low Pass Filter that provides a filtered voltage output, Vac passed to the voltage-controlled current source (VCCS) of the first voltage to current converter, details of which are depicted in FIG. 14. Also depicted in FIG. 14 is an optional, isolated Current Sensing output, which monitors the magnitude of the ac current passing through the human subject for safety considerations. Voltages are obtained from the human subject through the V+, V− electrodes. R_Body represents the resistance of the body between the V+, V− electrodes. U2 and U3 in this figure and U5 in FIG. 15 are transconductance amplifiers providing current outputs.

FIG. 15 depicts details of the second voltage to current converter. The V+, V− voltages are combined in the U4 amplifier and the differential voltage output passed to the U5 voltage to current converter, which converts the differential voltage into a current and adds a bias equal to that stripped out of the voltage signal at the AD5933 VOUT terminal. The resulting current (Current Out) is fed to the VIN and RFB terminals of the AD5933 circuit 650.

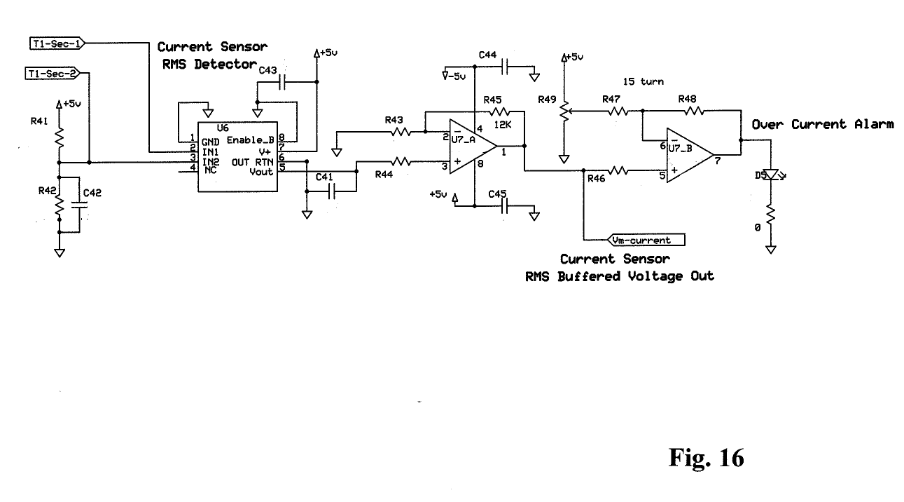

FIG. 16 provides details of a Current Sensor RMS Detector connected across the output side of the T1 transformer in the FIG. 14. As configured, it provides an RMS Buffered Voltage output that can be tapped by a monitoring circuit such as an input of the device controller 80. It also powers an Over Current Alarm in the form of a light source diode D5.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A device to monitor tissue hydration of a human subject, the device comprising:
at least four electrodes capable of physically adhering and electrically coupling to the human subject; and
circuitry coupled to four of the at least four electrodes to measure a bioelectric tissue impedance of the patient at a frequency of less than fifteen kilohertz. (<15 kHz).

2. The device of claim 1 wherein the circuitry is configured to determine extracellular fluid level status of the human subject based upon tissue impedance measured at a single frequency of less than fifteen kilohertz.

3. The device of claim 2, wherein the circuitry includes a first voltage to current subcircuit generating a current to be applied to the human subject between a first pair of the four electrodes and a second voltage to current subcircuit connected across a second pair of the four electrodes to be located on the subject between the first pair of electrodes to generate a single current output.

4. The device of claim 2, wherein single frequency is about 5 kHz.

5. The device of claim 1 wherein the circuitry includes:
a microprocessor controller;
an AD5933 Impedance Converter, Network Analyzer or equivalent circuit connected with the microprocessor controller; and
a Tetrapolar Analog Front End Patient Interface or equivalent circuit to interface the AD5933 Impedance Converter, Network Analyzer or equivalent circuit with the human subject and convert the bipolar impedance operation of the AD5933 Impedance Converter, Network Analyzer or equivalent into a tetrapolar operation on the human subject.

6. A method of operating the device of claim 1 to monitor extracellular fluid status of a human subject comprising the steps of:
connecting four of the electrodes in a linear arrangement to the skin of the human subject;
generating an oscillating voltage signal having a frequency of less than 15 kHz.;
removing a dc bias from the oscillating voltage signal;
converting the oscillating voltage signal into an oscillating current having a frequency of less than 15 kHz;
passing the oscillating current through the human subject between a first pair of the electrodes;
sampling voltages from the human subject through a second pair of electrodes positioned between the first pair of electrodes on the human subject;
generating a differential voltage signal from sampled voltages;
converting the differential voltage signal into an alternating current;
adding to the alternating current a constant bias equivalent to the dc bias removed from the oscillating voltage signal to provide a current output;
determining from the current output one or more biometric impedance values for the human subject.

7. The method of claim 6 wherein the steps after the connecting step are performed with the human subject undergoing dialysis.

8. A method of monitoring a human subject, the method comprising:
adhering to skin of the human subject four spaced apart electrodes in a linear array;
passing an oscillating current having a frequency of less that fifteen kilohertz through the patient between an outermost pair of the four electrodes;
sensing voltage levels from the human subject through an innermost pair of the four electrodes;
calculating a bioelectric impedance value for the human subject from the sensed voltage levels; and
outputting the biometric impedance value to a human interface device.

9. The method of claim 8 wherein the adhering step comprises attaching the four electrodes to a limb of the human subject and wherein the remaining steps of claim 5 are performed with the human subject undergoing dialysis.
